(19) United States
(12) Patent Application Publication
(10) Pub. No.: US 2007/0166721 A1
(43) Pub. Date: Jul. 19, 2007

(54) Fluidic circuits, methods and apparatus for use of whole blood samples in colorimetric assays

(76) Inventors: Brigitte C. Phan, Irvine, CA (US); Amethyst H. Lam, Irvine, CA (US); James H. Coombs, Singapore (SG); John F. Gordon, Irvine, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/874,817

(22) Filed: Jun. 23, 2004

Related U.S. Application Data
(60) Provisional application No. 60/483,342, filed on Jun. 27, 2003.

(51) Int. Cl. C12Q 1/68; C12M 3/00

(52) U.S. Cl. 435/6; 435/287.2

(57) ABSTRACT

When the quantification of an analyte is based on a color change detected by a change in the amount of light transmitted or reflected, undiluted samples often saturate the detection range of the assay. Thus, very often, the sample needs to be diluted for reliable quantification. Disclosed are systems and method, including various configurations of fluidic circuits for us on an optical bio-disc, that advantageously allow the use of undiluted and/or whole blood samples for colorimetric assays on optical bio-disc is described.

This application claims priority under 35 U.S.C. §119(e) to U.S. Provisional Application Ser. No. 60/483,342, filed on Jun. 27, 2003, the disclosure of which is incorporated by reference herein in its entirety.

### BACKGROUND OF THE INVENTION

1. Field of the Invention

This invention relates in general to assays and, in particular, colorimetric assays.

2. Description of the Related Art

The detection and quantification of analytes in body fluids, such as blood, may be helpful for the diagnosis of diseases, elucidation of the pathogenesis, and for monitoring the response to drug treatment. Traditionally, diagnostic assays are performed in laboratories by trained technicians using complex apparatus. Performing these assays is usually time-consuming and costly. Thus, there is a need to make diagnostic assays and forensic assays of all types faster and more local to the end-user. Ideally, clinicians, patients, investigators, the military, other health care personnel, and consumers should be able to test themselves for the presence of certain risk factors or disease indicators in their systems, and to test for the presence of certain biological material at a crime scene or on a battlefield.

Commonly assigned U.S. Pat. No. 6,030,581 entitled “Laboratory in a Disk” issued Feb. 29, 2000 (the '581 patent) is hereby incorporated by reference in its entirety. The '581 patent discloses an apparatus that includes an optical disc, adapted to be read by an optical reader, which has a sector having a substantially self-contained assay system useful for localizing and detecting an analyte suspected of being in a sample. U.S. Pat. No. 5,993,665, issued Nov. 30, 1999 (the '665 patent) entitled “Quantitative Cell Analysis Methods Employing Magnetic Separation” discloses systems and methods for analysis of biological specimens in a fluid medium, where the specimens are rendered magnetically responsive by immuno-specific binding with ferromagnetic colloid. The '665 patent is hereby incorporated by reference in its entirety.

### SUMMARY OF THE INVENTION

In one embodiment, the invention relates to performing colorimetric and fluorescent assays on an optical analysis disc or optical bio-disc. Described herein are methods for preparing assays, methods for depositing the reagents used for the assays, discs for performing assays, fluidic circuits and detection systems.

Current diagnostic and biochemical tests may employ a substance (chromagen) that undergoes a detectable color development or change or fluorescent emission in the presence of the analyte of interest. The intensity of the color or fluorescence developed may be time dependent and proportional to the concentration of the analyte of interest. For calorimetric assays, the intensity of the color may be measured by optical density measurement at specific wavelengths using a spectrophotometer, for example.

In one embodiment, methods for quantifying the concentration of an analyte of interest in a biological sample on optical bio-discs uses colorimetric assays. Analytes may include, for example, glucose, cholesterol, and triglycerides. In one embodiment, reagents necessary for the assays are deposited or stored in the optical biodisc prior to the assay. Alternatively, the reagents may be placed in reservoirs in the biodisc. To perform the assay, the sample (preferably whole blood, but other types of fluids could also be used) is loaded into the bio-disc via a sample inlet or injection port. In one embodiment, after the sample is loaded, the port is sealed. The disc may then be rotated to allow mixing of reagents and the sample. Depending on the assay protocol, the bio-disc may be incubated at room temperature for a predetermined time, such as 3 to 7 minutes, for example. The optical disc reader may then be used to quantify the intensity of the color developed. After data collection and processing, the results of the assay are displayed on a computer monitor. It should be noted that many diagnostic calorimetric assays in clinical laboratories are carried out at 37 degrees Celsius to facilitate and accelerate color development. However, calorimetric assays may be performed at any temperature. For ease of operation, colorimetric assays performed on optical discs may be optimized to run at ambient temperature. The optimization includes selection of enzyme sources, enzymes concentrations, and sample preparation.

Chromagen selection may be important in optimizing calorimetric assays for optical density measurements on bio-discs, since chromagens are detected at specific wavelengths. CD-R type disc readers or drives, for example, use chromagens that can be detected in the infrared region (750 nm to 800 nm). Other types of optical disc systems may be used in the invention including DVD, DVD-R, fluorescent, phosphorescent, and any other similar optical disc reader. The amplitude of optical density measurements depends at least upon the optical path length, the molar extinction coefficient of the chromagen and the concentration of the analyte of interest (Beer's law). To optimize the sensitivity of calorimetric assays on optical discs, several chromagens with high molar extinction coefficients at the wavelengths of interest have been identified and evaluated.

Chromagens suitable for colorimetric assays on CD-R type optical discs include, but are not limited to, N,N′-Bis(2-hydroxy-3-sulfopropyl)tolidine, disodium salt (SAT-3), N—(Carboxymethylaminocarbonyl)-4,4′-bis(dimethylamino)-diphenylamine sodium salt (DA-64), 2,2′-azino-dimethylthiozoline-6-sulfonate (ABTS), Trinder's reagents N-Ethyl-N-(2-hydroxy-3-sulfopropyl)₃-methylaniline, sodium salt, dihydrate (TOOS) with the coupling reagent 3-(N-Methyl-N-phenylamino)-6-aminobenzenesulfonic acid, and sodium salt (NCP-11).

A criterion that defines a good diagnostic assay is the ease by which one performs the assay. For colorimetric assays on optical bio-discs, all reagents necessary for the assay may be immobilized on the disc prior to the assay. There are several methods that can be used for reagent deposition. They include air or vacuum evaporation, enzyme immobilization by chemical linkage, lyophilization, or reagent deposition or printing on a suitable medium or reagent matrix material such as, for example, filter paper or membrane strips. All of the above methods have been tested on bio-discs. The preferred method has been found to be depositing one or more reagents on a matrix material or membrane strips because reagent stability for several weeks or months is preserved.

Alternatively, buffers, liquid reagents, or reagents dissolved in buffer may be pre-loaded into one or more solvent, buffer or reagent reservoirs in the bio-disc. The reagent reservoirs are in fluid communication with one or more mixing channels through a reagent channel that prevents the reagents from flowing through unless there is an external force that causes the reagents to flow through the reagent channel. The external force may come from centrifugal force generated by rotation of the disc. The amount of centrifugal force needed to release the reagents from their reservoirs may be controlled by the physical characteristics of the reagent channel such that different reagents may be released into the mixing chamber at different times by controlling the rotation speed of the disc. Examples of embodiments of fluidic circuits for use in the invention are described and illustrated in conjunction with FIGS. 22, 23A, 23B, 24, and 25.

The selection of membrane strips or matrix material for reagent deposition may be central in the success of the assay. Membrane strips are traditionally used in dipstick or lateral flow assays, where all of the chemistry occurs on a solid phase. However, in one embodiment, for colorimetric assays on optical analysis discs or biodiscs, the chemistry between the sample and the reagents occurs in solution. For this reason, the use of membrane strips in calorimetric assays on bio-discs is rather unique. Further, instead of using nitrocellulose membranes that are normally used in lateral flow assays, the membrane strips chosen for reagent deposition in colorimetric assays preferably have a good absorbing capacity to accommodate the volume of reagent deposited, while retaining good release efficiency. A membrane strip with good release efficiency allows the reagents to be released from the storage medium (membrane strip or matrix material) into solution as soon as a buffer, solvent or the sample flows through the matrix material, where they effectively catalyze the desired reactions. This allows for the color development from the reaction to be homogenous throughout the reaction or analysis chamber. The membrane strips for reagent deposition can be prepared independently of the discs and easily deposited within the disc during disc assembly. Numerous membrane strips have been tested for this particular function. One preferred membrane strip for reagent deposition is a hydrophilic polyethersulfone membrane of pore size approximately 0.2 um or larger.

In lab-based colorimetric assays, the concentrations of unknown samples were normally derived from calibrators or solutions with known concentrations. The use of calibrators necessitated additional preparation steps, which were more time-consuming and error prone. On optical bio-discs, calibrators in calorimetric assays may be replaced by calibration bars, which express the concentrations of the calibrators in terms of the relative amount of light transmitted or reflected. The calibration bars could be created either in the software or directly on the disc. The creation of calibration bars may reduce the assay time significantly and makes the assay much more user friendly. Alternatively, a calibration curve or points may be used to determine the concentration of analyte by analyzing for example a reagent blank analysis chamber and a chamber having a pre-determined amount of analyte as described below in conjunction with FIG. 24.

According to one aspect of the invention, there are provided detection methods for quantifying the concentration of an analyte of interest in a biological sample on the bio-discs. The detection includes directing a beam of electromagnetic energy from a disc drive toward an analysis chamber and analyzing electromagnetic energy returned from or transmitted through the capture field.

In one embodiment, the optical density change in calorimetric assays can be quantified by the optical disc reader in two related ways. These include measuring the change in light either reflected or transmitted. The disc may be referred to as reflective, transmissive, or some combination of reflective and transmissive. In a reflective disc, an incident light beam is focused onto the disc (typically at a reflective surface where information is encoded), reflected, and returned through optical elements to a detector on the same side of the disc as the light source. In a transmissive disc, light passes through the disc (or portions thereof) to a detector on the other side of the disc from the light source. In a transmissive portion of a disc, some light may also be reflected and detected as reflected light. Different detection systems are used for different types of bio-discs (top versus bottom detector).

The conversion of data captured by the CD reader into meaningful concentration units is mediated via data processing software specific for the assay of interest.

The apparatus and methods in embodiments of the invention can be designed for use by an end-user, inexpensively, without specialized expertise and expensive equipment. The system can be made portable, and thus usable in remote locations where traditional diagnostic equipment may not generally be available.

Alternatively, fluorescent assays can be carried out to quantify the concentration of one or more analytes of interest in a biological sample on the optical discs. In this case, the energy source in the disc drive preferably has a wavelength controllable light source and a detector that is or can be made specific to several wavelengths. Alternatively, a disc drive can be made with a specific light source and detector to produce a dedicated device, in which case the source may only need fine-tuning.

A bio-disc drive assembly or reader may be employed to rotate the disc, read and process any encoded information stored on the disc, and analyze the samples in the flow channel of the bio-disc. The bio-disc drive is thus provided with a motor for rotating the bio-disc, a controller for controlling the rate of rotation of the disc, a processor for processing return signals from the disc, and an analyzer for analyzing the processed signals. The drive may include software specifically developed for performing the assays disclosed herein.

The rotation rate of the motor is controlled to achieve the desired rotation of the disc. The bio-disc drive assembly may also be utilized to write information to the bio-disc either before or after the test material in the flow channel and target or capture zone is interrogated by the read beam of the drive and analyzed by the analyzer. The bio-disc may include encoded information for controlling the rotation rate of the disc, providing processing information specific to the type of test to be conducted, and for displaying the results on a display monitor associated with the bio-drive in accordance with the assay methods relating hereto.

One aspect of the present invention includes a method for performing an assay, comprising introducing a biological sample into a channel or reservoir in a bio-optical disk, wherein the bio-optical disk includes data or program information relevant to conducting or interpreting an assay for an analyte; contacting the sample with one or more reagents that produce a first colorimetric signal in the presence of analyte in the sample; contacting the one or more reagents with a species that interacts with one or more of the reagents in competition with any analyte in the sample, wherein any colorimetric signal produced as a result of the presence of the species is spectrally distinguishable from the first calorimetric signal; and measuring the first colorimetric signal to quantitate the amount of analyte, if any, in the sample. In one embodiment, the species produces a second colorimetric signal in cooperation with the reagents, further comprising measuring the second colorimetric signal and comparing the magnitude thereof with the first calorimetric signal. In another embodiment, the species produces a second colorimetric signal that is largely or wholly outside of a spectral range of sensitivity of a detector, such that the measuring step primarily or wholly involves measuring only the first colorimetric signal. Preferably, the method is performed in a disk drive and the species does not produce a signal that is substantially measured by the disk drive, such as either no calorimetric signal or one outside the spectral sensitivity range in which the disk drive is designed to operate. Preferably, one or more of the contacting steps is performed by moving fluid in the disk by spinning the disk at a predetermined speed. In one embodiment, the disk includes computer-readable information relative to calibration. In another embodiment, the disk includes computer-readable information that controls the performance of at least one aspect of the method, wherein the method is performed in a disk drive.

### BRIEF DESCRIPTION OF THE DRAWINGS

FIG. 1 is a pictorial representation of a bio-disc system according to the invention;

FIG. 2 is an exploded perspective view of a reflective bio-disc as utilized in conjunction with the invention;

FIG. 3 is a top plan view of the disc shown in FIG. 2;

FIG. 4 is a perspective view of the disc illustrated in FIG. 2 with cut-away sections showing the different layers of the disc;

FIG. 5 is an exploded perspective view of a transmissive bio-disc as employed in conjunction with the invention;

FIG. 6 is a perspective view representing the disc shown in FIG. 5 with a cut-away section illustrating the functional aspects of a semi-reflective layer of the disc;

FIG. 7 is a graphical representation showing the relationship between thickness and transmission of a thin gold film;

FIG. 8 is a top plan view of the disc shown in FIG. 5;

FIG. 9 is a perspective view of the disc illustrated in FIG. 5 with cut-away sections showing the different layers of the disc including the type of semi-reflective layer shown in FIG. 6;

FIG. 10 is a perspective and block diagram representation illustrating the system of FIG. 1 in more detail;

FIG. 11 is a partial cross sectional view taken perpendicular to a radius of the reflective optical bio-disc illustrated in FIGS. 2, 3, and 4 showing a flow channel formed therein;

FIG. 12 is a partial cross sectional view taken perpendicular to a radius of the transmissive optical bio-disc illustrated in FIGS. 5, 8, and 9 showing a flow channel formed therein and a top detector;

FIG. 13 is a partial longitudinal cross sectional view of the reflective optical bio-disc shown in FIGS. 2, 3, and 4 illustrating a wobble groove formed therein;

FIG. 14 is a partial longitudinal cross sectional view of the transmissive optical bio-disc illustrated in FIGS. 5, 8, and 9 showing a wobble groove formed therein and a top detector;

FIG. 15 is a view similar to FIG. 11 showing the entire thickness of the reflective disc and the initial refractive property thereof;

FIG. 16 is a view similar to FIG. 12 showing the entire thickness of the transmissive disc and the initial refractive property thereof;

FIG. 17 is an illustration of a reaction sequence for a quantifiable reaction and its competing reaction;

FIG. 18 is a graphical representation of an absorption spectra of Trider's reagents with a coupling reagent NCP-11;

FIG. 19 is a graphical representation of an absorption spectra of Trider's reagents with a coupling reagent 4AAP;

FIG. 20 is a top plan view of the optical disc of the invention including fluidic circuits with a reagent release area containing a reagent release material;

FIG. 21 shows various steps for analyte-dependent generation of hydrogen peroxide;

FIG. 22 is a schematic representation of a fluidic circuit having a single solvent chamber for analysis of a whole blood sample;

FIG. 23A is a schematic illustration of a fluidic circuit having two solvent chambers for analysis of a whole blood sample;

FIG. 23B is a schematic illustration of an alternate embodiment of the fluidic circuit of FIG. 23A;

FIG. 24 is a schematic representation of a fluidic circuit having multiple analysis fluidic circuits for quatitation of analytes in a whole blood sample; and

FIG. 25 is a top plan view of an optical bio-disc having the fluidic circuits shown in FIGS. 22, 23A, and 24.

### DETAILED DESCRIPTION OF THE PREFERRED EMBODIMENT

Embodiments of the invention will now be described with reference to the accompanying Figures, wherein like numerals refer to like elements throughout. The terminology used in the description presented herein is not intended to be interpreted in any limited or restrictive manner, simply because it is being utilized in conjunction with a detailed description of certain specific embodiments of the invention. Furthermore, embodiments of the invention may include several novel features, no single one of which is solely responsible for its desirable attributes or which is essential to practicing the inventions herein described.

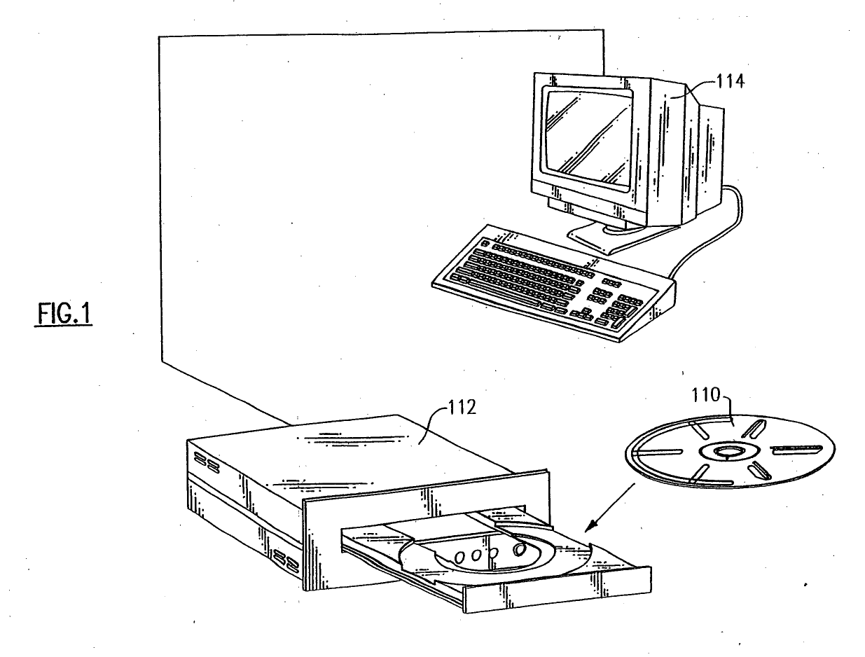

FIG. 1 is a perspective view of an optical bio-disc 110 configured to conduct the cell counts and differential cell counts disclosed herein. The present optical bio-disc 110 is shown in conjunction with an optical disc drive 112 and a display monitor 114. Further details relating to this type of disc drive and disc analysis system are disclosed in commonly U.S. patent application Ser. No. 10/008,156 entitled “Disc Drive System and Methods for Use with Bio-discs” filed Nov. 9, 2001 and U.S. patent Application Ser. No. 10/043,688 entitled “Optical Disc Analysis System Including Related Methods For Biological and Medical Imaging” filed Jan. 10, 2002, both of which are herein incorporated by reference.

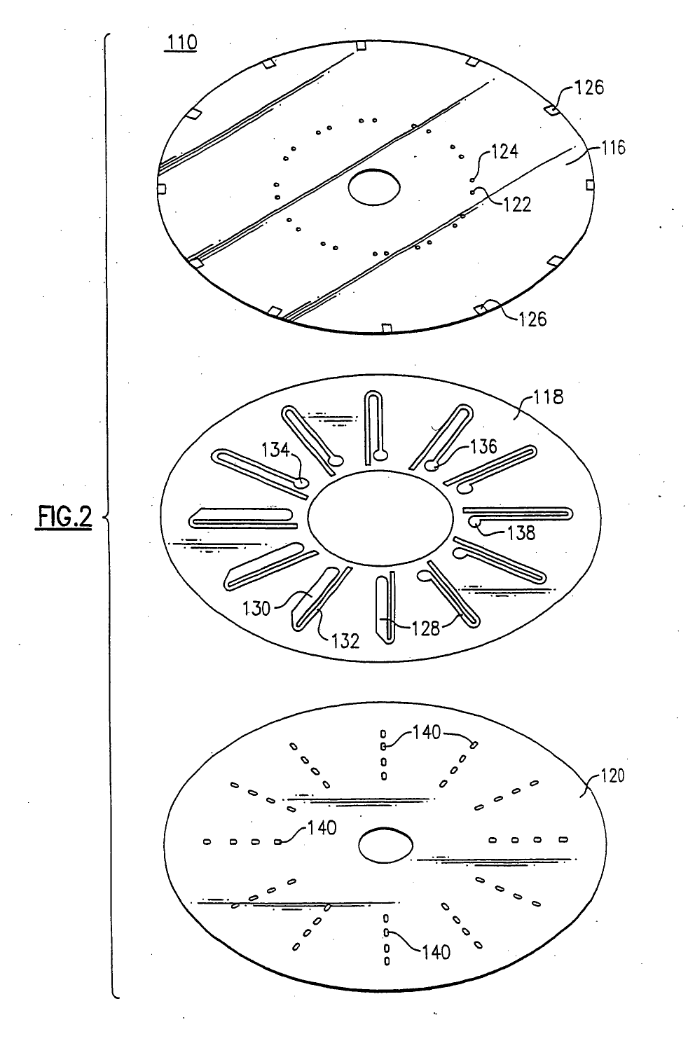

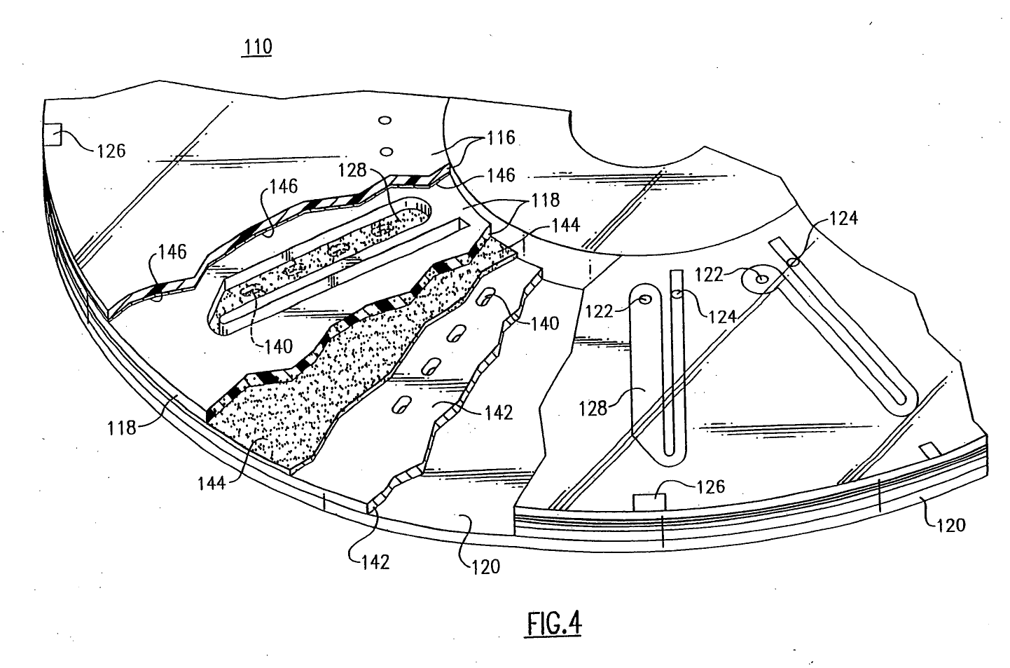

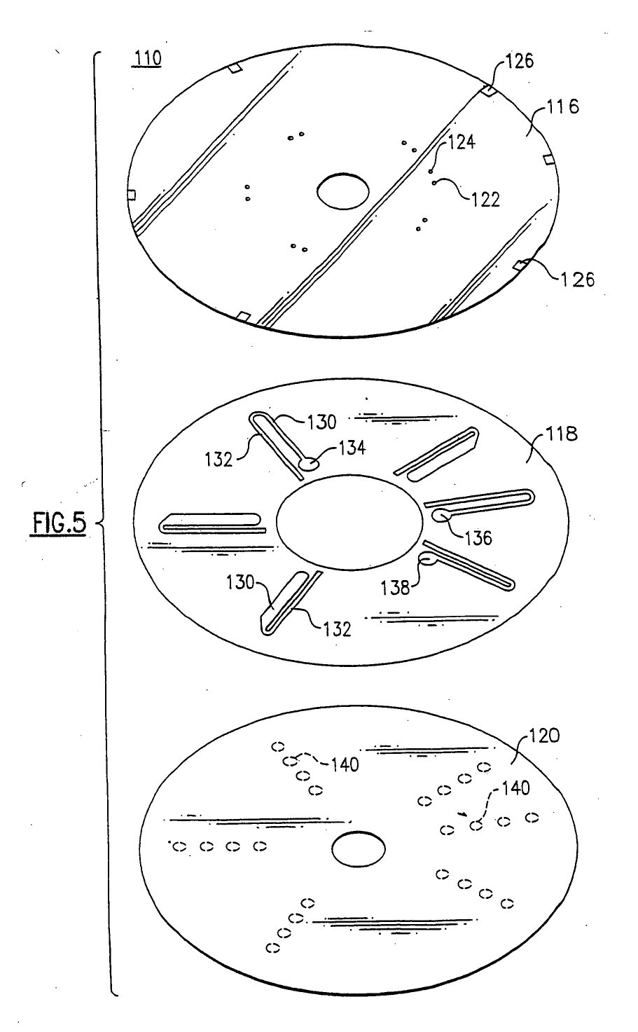

FIG. 2 is an exploded perspective view of the principal structural elements of one embodiment of the optical bio-disc 110. FIG. 2 is an example of a reflective zone optical bio-disc 110 (hereinafter “reflective disc”) that may be used in the invention. The principal structural elements include a cap portion 116, an adhesive member or channel layer 118, and a substrate 120. The cap portion 116 includes one or more inlet ports 122 and one or more vent ports 124. The cap portion 116 may be formed from polycarbonate and is preferably coated with a reflective surface 146 (FIG. 4) on the bottom thereof as viewed from the perspective of FIG. 2. In the preferred embodiment, trigger marks or markings 126 are included on the surface of the reflective layer 142 (FIG. 4). Trigger markings 126 may include a clear window in multiple, or all, layers of the bio-disc, an opaque area, or a reflective or semi-reflective area encoded with information that sends data to a processor 166, as shown FIG. 10, that in turn interacts with the operative functions of the interrogation or incident beam 152, FIGS. 6 and 10.

The second element shown in FIG. 2 is an adhesive member or channel layer 118 having fluidic circuits 128 or U-channels formed therein. The fluidic circuits 128 are formed by stamping or cutting the membrane to remove plastic film and form the shapes as indicated. Each of the fluidic circuits 128 includes a flow channel 130 and a return channel 132. Some of the fluidic circuits 128 illustrated in FIG. 2 include a mixing chamber 134. Two different types of mixing chambers 134 are illustrated. The first is a symmetric mixing chamber 136 that is symmetrically formed relative to the flow channel 130. The second is an off-set mixing chamber 138. The off-set mixing chamber 138 is formed to one side of the flow channel 130 as indicated.

The third element illustrated in FIG. 2 is a substrate 120 including target or capture zones 140. The substrate 120 is preferably made of polycarbonate and has a reflective layer 142 deposited on the top thereof, FIG. 4. The target zones 140 are formed by removing the reflective layer 142 in the indicated shape or alternatively in any desired shape. Alternatively, the target zone 140 may be formed by a masking technique that includes masking the target zone 140 area before applying the reflective layer 142. The reflective layer 142 may be formed from a metal such as aluminum or gold.

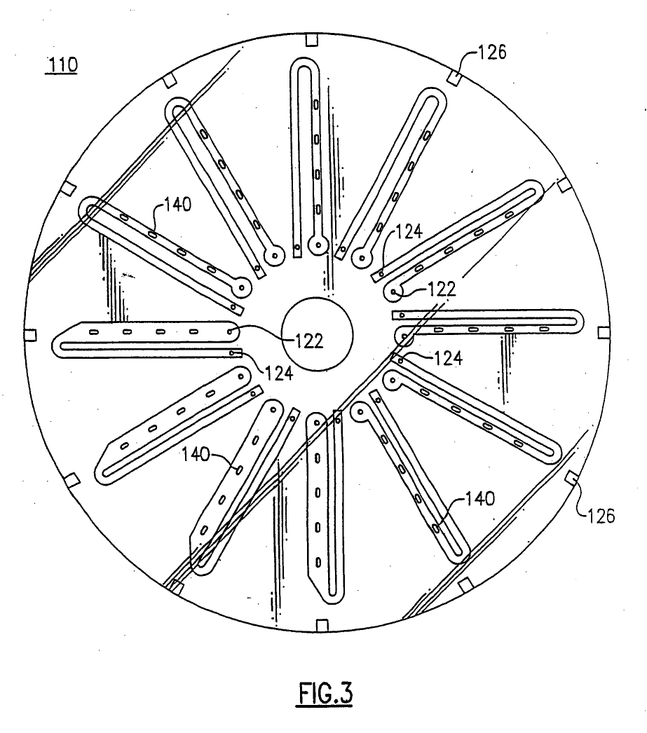

FIG. 3 is a top plan view of the optical bio-disc 110 illustrated in FIG. 2 with the reflective layer 142 on the cap portion 116 shown as transparent to reveal the fluidic circuits 128, the target zones 140, and trigger markings 126 situated within the disc.

FIG. 4 is an enlarged perspective view of the reflective zone type optical bio-disc 110 according to one embodiment of the invention. This view includes a portion of the various layers thereof, cut away to illustrate a partial sectional view of each principal layer, substrate, coating, or membrane. FIG. 4 shows the substrate 120 that is coated with the reflective layer 142. An active layer 144 is applied over the reflective layer 142. In the preferred embodiment, the active layer 144 may be formed from polystyrene. Alternatively, polycarbonate, gold, activated glass, modified glass, or modified polystyrene, for example, polystyrene-co-maleic anhydride, may be used. In addition, hydrogels can be used. Alternatively as illustrated in this embodiment, the plastic adhesive member 118 is applied over the active layer 144. The exposed section of the plastic adhesive member 118 illustrates the cut out or stamped U-shaped form that creates the fluidic circuits 128. The final principal structural layer in this reflective zone embodiment of the present bio-disc is the cap portion 116. The cap portion 116 includes the reflective surface 146 on the bottom thereof. The reflective surface 146 may be made from a metal such as aluminum or gold.

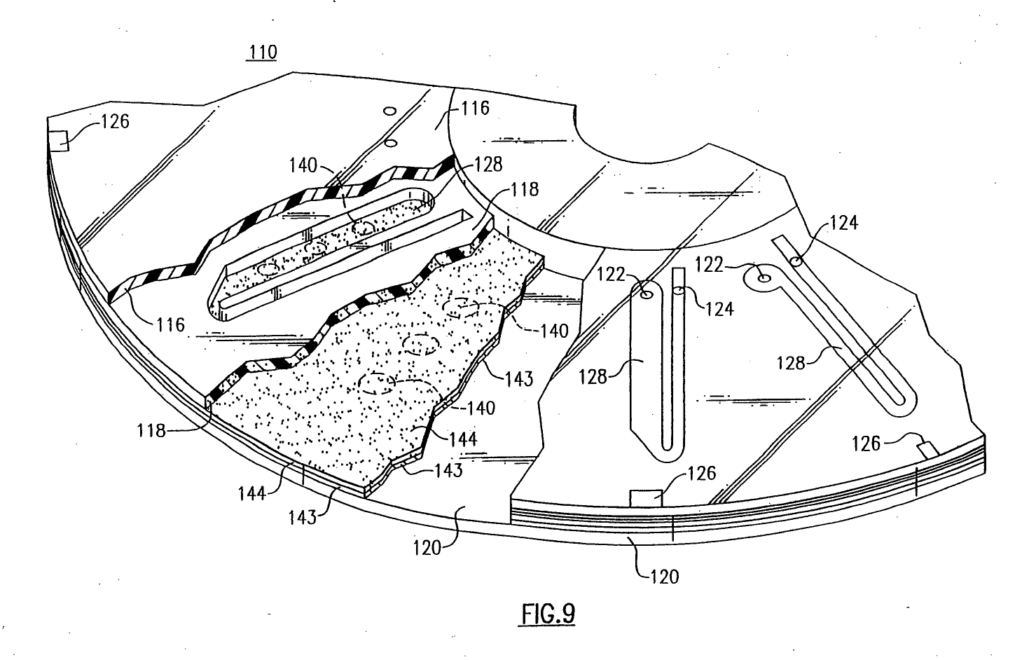

Referring now to FIG. 5, there is shown an exploded perspective view of the principal structural elements of a transmissive type of optical bio-disc 110 according to the invention. The principal structural elements of the transmissive type of optical bio-disc 110 similarly include the cap portion 116, the adhesive or channel member 118, and the substrate 120 layer. The cap portion 116 includes one or more inlet ports 122 and one or more vent ports 124. The cap portion 116 may be formed from a polycarbonate layer. Optional trigger markings 126 may be included on the surface of a thin semi-reflective layer 143, as illustrated in FIGS. 6 and 9. Trigger markings 126 may include a clear window in all three layers of the bio-disc, an opaque area, or a reflective or semi-reflective area encoded with information that sends data to the processor 166, FIG. 10, which in turn interacts with the operative functions of the interrogation beam 152, FIGS. 6 and 10.

The second element shown in FIG. 5 is the adhesive member or channel layer 118 having fluidic circuits 128 or U-channels formed therein. The fluidic circuits 128 are formed by stamping or cutting the membrane to remove plastic film and form the shapes as indicated. Each of the fluidic circuits 128 includes the flow channel 130 and the return channel 132. Some of the fluidic circuits 128 illustrated in FIG. 5 include the mixing chamber 134. Two different types of mixing chambers 134 are illustrated. The first is the symmetric mixing chamber 136 that is symmetrically formed relative to the flow channel 130. The second is the off-set mixing chamber 138. The off-set mixing chamber 138 is formed to one side of the flow channel 130 as indicated.

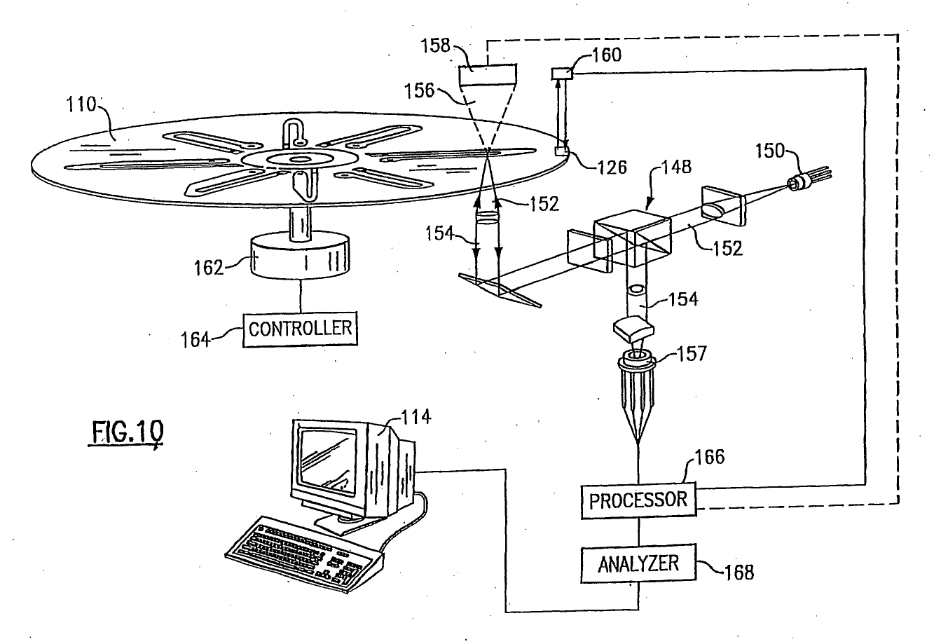

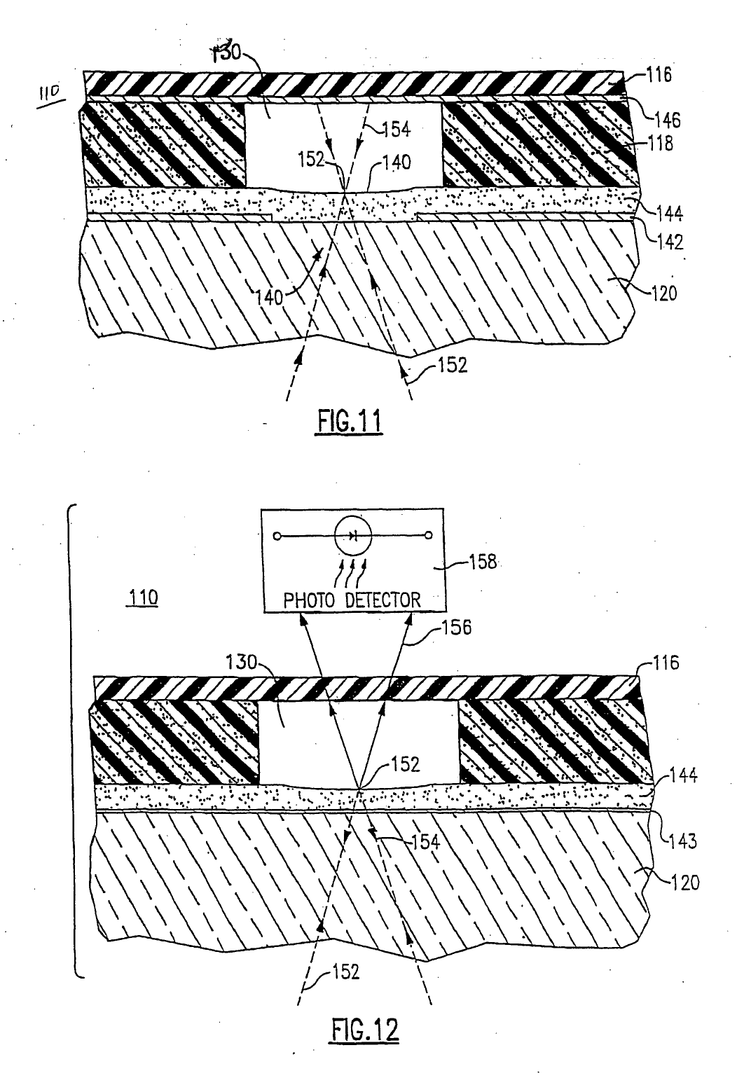

The third element illustrated in FIG. 5 is the substrate 120 which may include the target or capture zones 140. The substrate 120 is preferably made of polycarbonate and has the thin semi-reflective layer 143 deposited on the top thereof, FIG. 6. The semi-reflective layer 143 associated with the substrate 120 of the disc 110 illustrated in FIGS. 5 and 6 may be significantly thinner than the reflective layer 142 on the substrate 120 of the reflective disc 110 illustrated in FIGS. 2, 3 and 4. The thinner semi-reflective layer 143 allows for some transmission of the interrogation beam 152 through the structural layers of the transmissive disc as shown in FIGS. 6 and 12. The thin semi-reflective layer 143 may be formed from a metal such as aluminum or gold.

FIG. 6 is an enlarged perspective view of the substrate 120 and semi-reflective layer 143 of the transmissive embodiment of the optical bio-disc 110 illustrated in FIG. 5. The thin semi-reflective layer 143 may be made from a metal such as aluminum or gold. In the preferred embodiment, the thin semi-reflective layer 143 of the transmissive disc illustrated in FIGS. 5 and 6 is approximately 10-300 Å thick and does not exceed 400 Å. This thinner semi-reflective layer 143 allows a portion of the incident or interrogation beam 152 to penetrate and pass through the semi-reflective layer 143 to be detected by a top detector 158, FIGS. 10 and 12, while some of the light is reflected or returned back along the incident path.

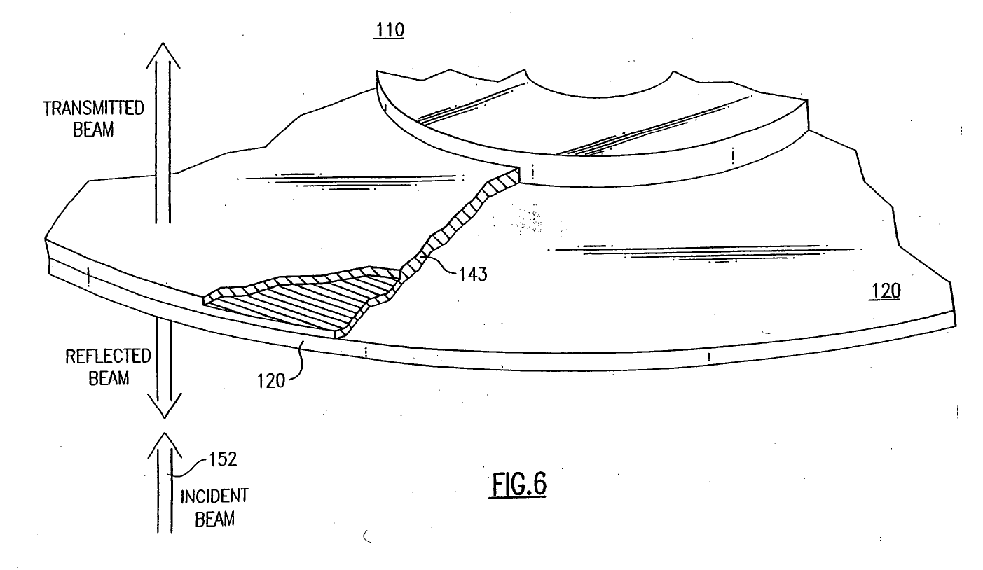

FIG. 7 provides a graphical representation of the inverse relationship of the reflective and transmissive nature of the thin semi-reflective layer 143 based upon the thickness of the gold. Reflective and transmissive values used in the graph illustrated in FIG. 7 are absolute values.

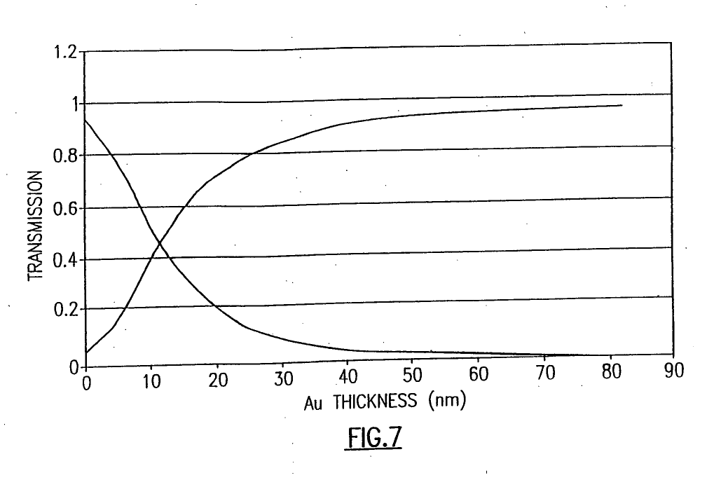

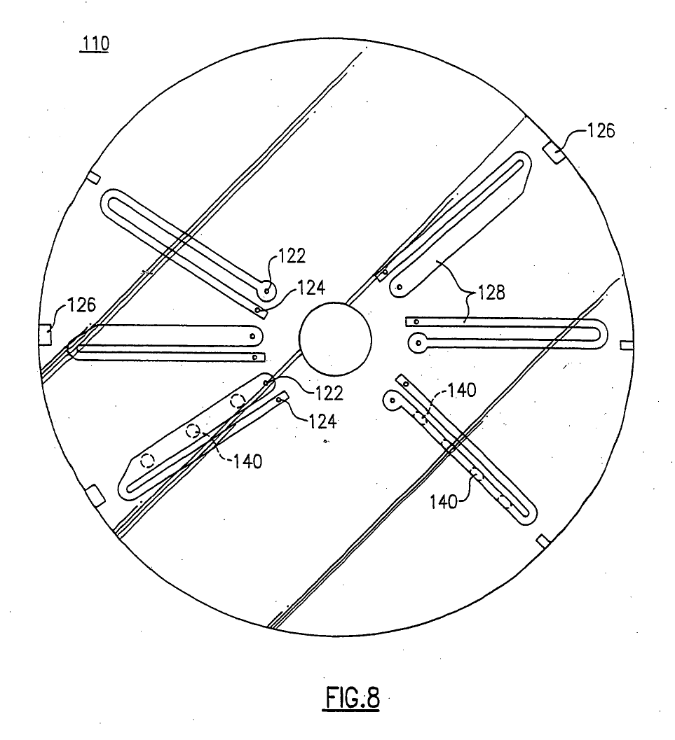

With reference next to FIG. 8, there is shown a top plan view of the transmissive type optical bio-disc 110 illustrated in FIGS. 5 and 6 with the transparent cap portion 116 revealing the fluidic channels, the trigger markings 126, and the target zones 140 as situated within the disc.

FIG. 9 is an enlarged perspective view of the optical bio-disc 110 according to the transmissive disc embodiment of the invention. The disc 110 is illustrated with a portion of the various layers thereof cut away to show a partial sectional view of each principal layer, substrate, coating, or membrane. FIG. 9 illustrates a transmissive disc format with the clear cap portion 116, the thin semi-reflective layer 143 on the substrate 120, and trigger markings 126. In this embodiment, trigger markings 126 include opaque material placed on the top portion of the cap. Alternatively the trigger marking 126 may be formed by clear, non-reflective windows etched on the thin reflective layer 143 of the disc, or any mark that absorbs or does not reflect the signal coming from the trigger detector 160, FIG. 10. FIG. 9 also shows the target zones 140 formed by marking the designated area in the indicated shape or alternatively in any desired shape. Markings to indicate target zone 140 may be made on the thin semi-reflective layer 143 on the substrate 120 or on the bottom portion of the substrate 120 (under the disc). Alternatively, the target zones 140 may be formed by a masking technique that includes masking all, or a portion, of the entire thin semi-reflective layer 143 except the target zones 140. In this embodiment, target zones 140 may be created by silk screening ink onto the thin semi-reflective layer 143. In the transmissive disc format illustrated in FIGS. 5, 8, and 9, the target zones 140 may alternatively be defined by address information encoded on the disc. In this embodiment, target zones 140 do not include a physically discernable edge boundary.

With continuing reference to FIG. 9, an active layer 144 is illustrated as applied over the thin semi-reflective layer 143. In the preferred embodiment, the active layer 144 is a 10 to 200 μm thick layer of 2% polystyrene. Alternatively, polycarbonate, gold, activated glass, modified glass, or modified polystyrene, for example, polystyrene-co-maleic anhydride, may be used. In addition, hydrogels can be used. As illustrated in this embodiment, the plastic adhesive member 118 is applied over the active layer 144. The exposed section of the plastic adhesive member 118 illustrates the cut out or stamped U-shaped form that creates the fluidic circuits 128.

The final principal structural layer in this transmissive embodiment of the present bio-disc 110 is the clear, non-reflective cap portion 116 that includes inlet ports 122 and vent ports 124.

Referring now to FIG. 10, there is a representation in perspective and block diagram illustrating optical components 148, a light source 150 that produces the incident or interrogation beam 152, a return beam 154, and a transmitted beam 156. In the case of the reflective bio-disc illustrated in FIG. 4, the return beam 154 is reflected from the reflective surface 146 of the cap portion 116 of the optical bio-disc 110. In this reflective embodiment of the present optical bio-disc 110, the return beam 154 is detected and analyzed for the presence of signal elements by a bottom detector 157. In the transmissive bio-disc format, on the other hand, the transmitted beam 156 is detected, by a top detector 158, and is also analyzed for the presence of signal elements. In the transmissive embodiment, a photo detector may be used as a top detector 158.

FIG. 10 also shows a hardware trigger mechanism that includes the trigger markings 126 on the disc and a trigger detector 160. The hardware triggering mechanism is used in both reflective bio-discs (FIG. 4) and transmissive bio-discs (FIG. 9). The triggering mechanism allows the processor 166 to collect data only when the interrogation beam 152 is on a respective target zone 140. Furthermore, in the transmissive bio-disc system, a software trigger may also be used. The software trigger uses the bottom detector to signal the processor 166 to collect data as soon as the interrogation beam 152 hits the edge of a respective target zone 140. FIG. 10 further illustrates a drive motor 162 and a controller 164 for controlling the rotation of the optical bio-disc 110. FIG. 10 also shows the processor 166 and analyzer 168 implemented in the alternative for processing the return beam 154 and transmitted beam 156 associated the transmissive optical bio-disc.

As shown in FIG. 11, there is presented a partial cross sectional view of the reflective disc embodiment of the optical bio-disc 110 according to the invention. FIG. 11 illustrates the substrate 120 and the reflective layer 142. As indicated above, the reflective layer 142 may be made from a material such as aluminum, gold or other suitable reflective material. In this embodiment, the top surface of the substrate 120 is smooth. FIG. 11 also shows the active layer 144 applied over the reflective layer 142. As also shown in FIG. 11, the target zone 140 is formed by removing an area or portion of the reflective layer 142 at a desired location or, alternatively, by masking the desired area prior to applying the reflective layer 142. As further illustrated in FIG. 11, the plastic adhesive member 118 is applied over the active layer 144. FIG. 11 also shows the cap portion 116 and the reflective surface 146 associated therewith. Thus when the cap portion 116 is applied to the plastic adhesive member 118 including the desired cutout shapes, flow channel 130 is thereby formed. As indicated by the arrowheads shown in FIG. 11, the path of the incident beam 152 is initially directed toward the substrate 120 from below the disc 110. The incident beam then focuses at a point proximate the reflective layer 142. Since this focusing takes place in the target zone 140 where a portion of the reflective layer 142 is absent, the incident continues along a path through the active layer 144 and into the flow channel 130. The incident beam 152 then continues upwardly traversing through the flow channel to eventually fall incident onto the reflective surface 146. At this point, the incident beam 152 is returned or reflected back along the incident path and thereby forms the return beam 154.

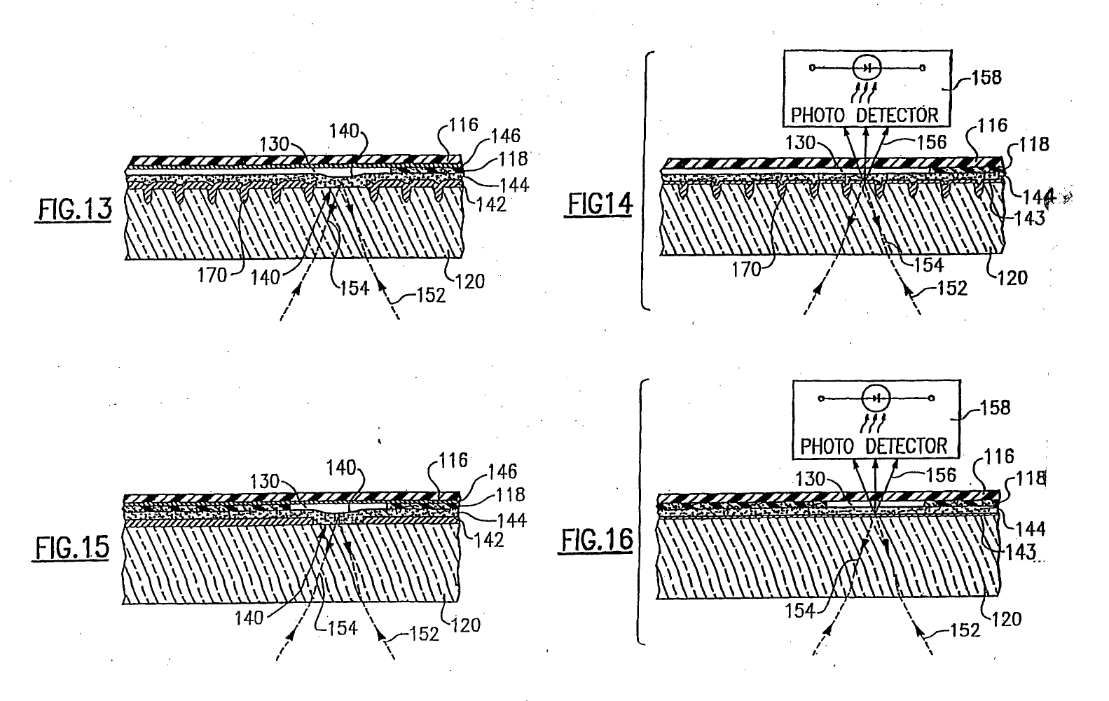

FIG. 12 is a partial cross sectional view of the transmissive embodiment of the bio-disc 110 according to the invention. FIG. 12 illustrates a transmissive disc format with the clear cap portion 116 and the thin semi-reflective layer 143 on the substrate 120. FIG. 12 also shows the active layer 144 applied over the thin semi-reflective layer 143. In the preferred embodiment, the transmissive disc has the thin semi-reflective layer 143 made from a metal such as aluminum or gold approximately 100-300 Angstroms thick and does not exceed 400 Angstroms. This thin semi-reflective layer 143 allows a portion of the incident or interrogation beam 152, from the light source 150, FIG. 10, to penetrate and pass upwardly through the disc to be detected by a top detector 158, while some of the light is reflected back along the same path as the incident beam but in the opposite direction. In this arrangement, the return or reflected beam 154 is reflected from the semi-reflective layer 143. Thus in this manner, the return beam 154 does not enter into the flow channel 130. The reflected light or return beam 154 may be used for tracking the incident beam 152 on pre-recorded information tracks formed in or on the semi-reflective layer 143 as described in more detail in conjunction with FIGS. 13 and 14. In the disc embodiment illustrated in FIG. 12, a physically defined target zone 140 may or may not be present. Target zone 140 may be created by direct markings made on the thin semi-reflective layer 143 on the substrate 120. These marking may be formed using silk screening or any equivalent method. In the alternative embodiment where no physical indicia are employed to define a target zone (such as, for example, when encoded software addressing is utilized) the flow channel 130 in effect may be employed as a confined target area in which inspection of an investigational feature is conducted.

FIG. 13 is a cross sectional view taken across the tracks of the reflective disc embodiment of the bio-disc 110 according to the invention. This view is taken longitudinally along a radius and flow channel of the disc. FIG. 13 includes the substrate 120 and the reflective layer 142. In this embodiment, the substrate 120 includes a series of grooves 170. The grooves 170 are in the form of a spiral extending from near the center of the disc toward the outer edge. The grooves 170 are implemented so that the interrogation beam 152 may track along the spiral grooves 170 on the disc. This type of groove 170 is known as a “wobble groove”. A bottom portion having undulating or wavy sidewalls forms the groove 170, while a raised or elevated portion separates adjacent grooves 170 in the spiral. The reflective layer 142 applied over the grooves 170 in this embodiment is, as illustrated, conformal in nature. FIG. 13 also shows the active layer 144 applied over the reflective layer 142. As shown in FIG. 13, the target zone 140 is formed by removing an area or portion of the reflective layer 142 at a desired location or, alternatively, by masking the desired area prior to applying the reflective layer 142. As further illustrated in FIG. 13, the plastic adhesive member 118 is applied over the active layer 144. FIG. 13 also shows the cap portion 116 and the reflective surface 146 associated therewith. Thus, when the cap portion 116 is applied to the plastic adhesive member 118 including the desired cutout shapes, the flow channel 130 is thereby formed.

FIG. 14 is a cross sectional view taken across the tracks of the transmissive disc embodiment of the bio-disc 110 according to the invention as described in FIG. 12, for example. This view is taken longitudinally along a radius and flow channel of the disc. FIG. 14 illustrates the substrate 120 and the thin semi-reflective layer 143. This thin semi-reflective layer 143 allows the incident or interrogation beam 152, from the light source 150, to penetrate and pass through the disc to be detected by the top detector 158, while some of the light is reflected back in the form of the return beam 154. The thickness of the thin semi-reflective layer 143 is determined by the minimum amount of reflected light required by the disc reader to maintain its tracking ability. The substrate 120 in this embodiment, like that discussed in FIG. 13, includes the series of grooves 170. The grooves 170 in this embodiment are also preferably in the form of a spiral extending from near the center of the disc toward the outer edge. The grooves 170 are implemented so that the interrogation beam 152 may track along the spiral. FIG. 14 also shows the active layer 144 applied over the thin semi-reflective layer 143. As further illustrated in FIG. 14, the plastic adhesive member or channel layer 118 is applied over the active layer 144. FIG. 14 also shows the cap portion 116 without a reflective surface 146. Thus, when the cap is applied to the plastic adhesive member 118 including the desired cutout shapes, the flow channel 130 is thereby formed and a part of the incident beam 152 is allowed to pass therethrough substantially unreflected.

FIG. 15 is a view similar to FIG. 11 showing the entire thickness of the reflective disc and the initial refractive property thereof. FIG. 16 is a view similar to FIG. 12 showing the entire thickness of the transmissive disc and the initial refractive property thereof. Grooves 170 are not seen in FIGS. 15 and 16 since the sections are cut along the grooves 170. FIGS. 15 and 16 show the presence of the narrow flow channel 130 that is situated perpendicular to the grooves 170 in these embodiments. FIGS. 13, 14, 15, and 16 show the entire thickness of the respective reflective and transmissive discs. In these figures, the incident beam 152 is illustrated initially interacting with the substrate 120 which has refractive properties that change the path of the incident beam as illustrated to provide focusing of the beam 152 on the reflective layer 142 or the thin semi-reflective layer 143.

In many applications, it may be desireable to perform quantitative assays with an undiluted volume of sample or whole blood sample. Without the dilution step or with use of whole blood, the number of steps in the assay may be reduced and the possibility of error may be minimized. In the case of a whole blood sample, using an undiluted sample may also minimize the possibility of blood cell lysis.

When the quantification of an analyte is based on a color change detected by a change in the amount of light transmitted or reflected, undiluted samples often saturate the detection range of the assay. Thus, very often, the sample needs to be diluted for reliable quantification. Following are systems and method that advantageously allow the use of undiluted and/or whole blood samples for colorimetric assays on optical bio-disc is described. The systems and methods described herein are applicable to a large number of quantitative assyas and are not limited to any one assay or Analyte.

In one embodiment, the assay is a competitive assay giving a quantitative signal. In this embodiment, both (1) reagents used for the quantitative assays of interest, referred to as the quantifiable reaction, and (2) reagents needed for a competing reaction are stored in a reagent release area in a fluidic circuit, either directly on the optical bio-disc or on a reagent release matrix. In one embodiment, the competing reaction uses the same substrate as the quantifiable reaction. In one embodiment, the end product of the competing reaction is not detectable at the wavelength of the optical drive light source or laser. The generation of the end product of the competing reaction will reduce proportionately the amount of end product that is detectable by the drive laser produced by a given amount of analyte.

In one embodiment, calorimetric assays using the optical bio-disc utilize chromagens, which when oxidized by horseradish peroxidase, for example, in the presence of hydrogen peroxide, generate products that are detectable in the infrared region, at around 780 nm wavelength. The quantifiable reaction consists of measuring the optical density of the colored oxidized chromagen using the optical disc reader. As discussed above, in the competing reaction, a different chromagen or other reactant, such as a catalase, may be used, which also utilizes the same substrate, hydrogen peroxide. The chromagen, or other reactant, in the competing reaction will compete for the same substrate, the oxidized chromagen, or other reaction product, however is not detectable by the optical disc reader.

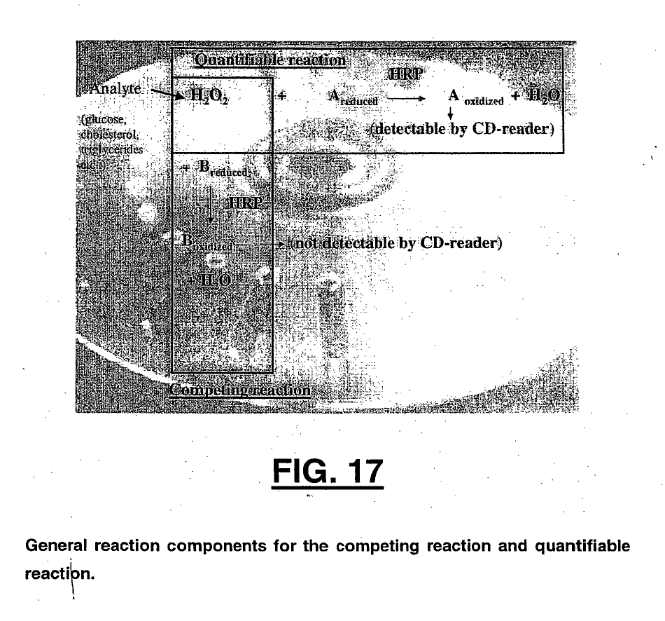

As shown in FIG. 17, in the quantifiable reaction, chromagen A, when oxidized by HRP in the presence of hydrogen peroxide H₂O₂ generates a product A_{oxidized} that is detectable by the optical disc reader or CD reader. In the competing reaction, chromagen B competitively uses the same substrate, hydrogen peroxide, to generate an oxidized form of B that is not detectable by the CD-reader or optical disc reader. The competing reaction thus reduces the amount of product that is detectable by the CD-reader. The saturation point (or the upper limit of the detection range) can be adjusted by varying the ratio of chromagens used in the competing and the quantifiable reactions, such that no dilution of the sample is required.

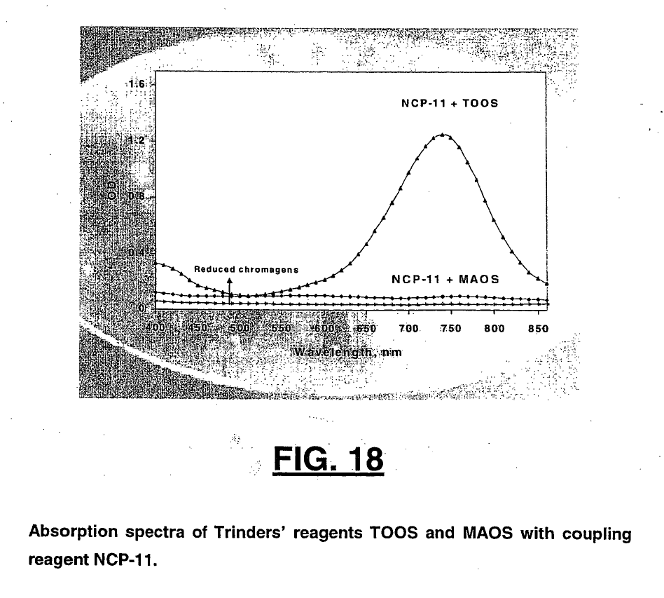

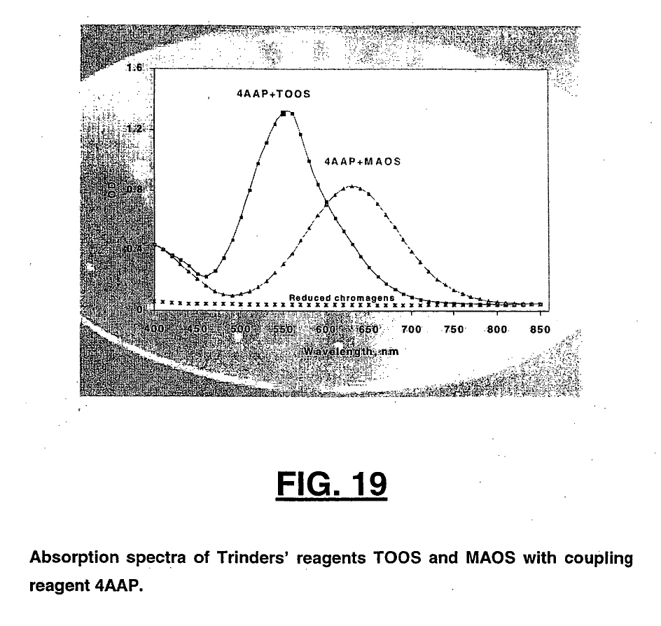

An exemplary system of quantifiable reaction and competing reaction employs the combination of Trinder's reagents N-Ethyl-N-(2-hydroxy-3-sulfopropyl)₃-methylaniline, sodium salt, dihydrate (TOOS) with the coupling reagent 3-(N-Methyl-N-phenylamino)-6-aminobenzenesulfonic acid, and sodium salt (NCP-11) in the quantifiable reaction and N-Ethyl-N-(2-hydroxy-3-sulfopropyl)3,5-dimethylaniline, sodium salt, monohydrate (MAOS) with the same coupling reagent NCP-11 in the competing reaction. As shown in FIG. 18, the absorption spectrum of TOOS and NCP-11 shows a clear peak at 750 nm, with a significant absorption signal at 780 nm. The absorption spectrum of MAOS and NCP-11, however, does not show any peak in the region scanned. Yet, when the Trinder's reagent MAOS was used in combination with a different coupling reagent 4-aminoantipyrine, the resulting product had an absorption peak at 630 nm, (FIG. 19) suggesting that the Trinder's reagent MAOS is a substrate for HRP and that the oxidized product resulting from the reaction of MAOS and NCP-11 does not have any absorption peak in the 400 to 800 nm region. Therefore, the use of Trinder's reagent MAOS and the coupling reagent NCP-11 in a competing reaction would be compatible with the combination of TOOS and NCP-11 in a quantifiable reaction. As would be apparent to one of skill in the art, similar systems could be developed using other chromagens without undue experimentation.

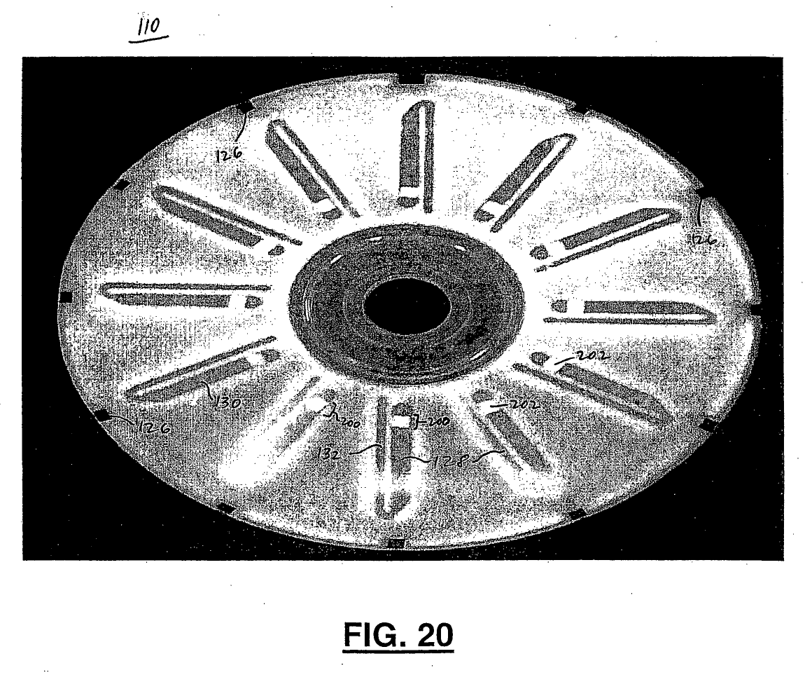

In one embodiment of the invention, reagents necessary for the quantifiable and competing reactions are immobilized on a reagent matrix material. The reagent matrix material may be a bio-membrane such as nitrocellulose or other membrane materials. The matrix material may be a partial matrix functioning as a padding wherein the bio membrane material only partially fills the reagent release area allowing fluid to flow though, over or around the membrane, or it may be a filling matrix such that the matrix fills the entire reagent release area such that fluid has to flow through the matrix. For each assay, the reagents may be stored in 3×5×0.3 mm membrane strips or pads, for example. The reagents may be loaded or deposited into the matrix material or pad manually with a pipettor, or by automatic applicators. The volume of reagents deposited on the strips may vary from 0.1 to 50 ul, for example. The pads may be placed within the reagent release area of the bio-disc at the time of assembly. In one embodiment, the thickness of the reagent strips or matrix material is such that they will fit securely within the channels of the bio-disc as illustrated in FIG. 20.

FIG. 20 is a top plan view of the transmissive disc having fluidic circuits 128 with a reagent release area 200. In the embodiment of FIG. 20, a matrix material 202 is placed in the reagent release area 200. The bio-disc illustrated in FIG. 20 may include the components of the discs described above in conjunction with FIGS. 5, 6, 7, 8, and 9, for example.

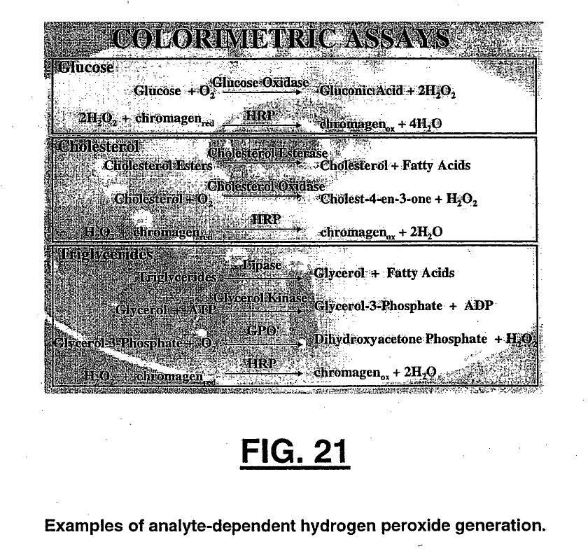

At the time of the assay, sample is injected into, or on to, the disc. The analyte of interest including, for example, glucose, cholesterol, triglycerides and glycerol-3-phosphate, undergoes a series of chemical reactions as shown in FIG. 21, leading ultimately to the production of hydrogen peroxide (H₂O₂). In one embodiment, both of the reagents, such as the Trinders' reagents discussed above, in the competing reaction and the quantifiable reaction competitively use the same substrate, with only the end product of the quantifiable reaction detected and quantified using the optical disc reader of the invention. The consumption of the analyte-dependent production of hydrogen peroxide by the competing but “silent reaction” allows for the detection of a much wider range of analyte concentrations.

With reference to FIG. 22, there is shown a schematic representation of a fluidic circuit for sample analysis. The exemplary fluidic circuit illustrated in FIG. 22 includes a solvent chamber 203 in fluid communication with a first vent channel 205 with a first vent port 206. Solvent or buffer is loaded into the solvent chamber 203 through a solvent inlet port 204. The solvent chamber 203 is in fluid communication with a reagent channel 207 having a reagent release area with a reagent matrix material 202. The matrix material 202 may be a filling matrix material that fills the reagent release area such that fluid has to flow through the matrix material to pass through the reagent channel 207. Alternatively, the matrix material 202 may be a partial matrix functioning as a padding which may only fill the reagent release area partially such that fluid is allowed to flow over or around the matrix material and through the matrix material 202 when fluid passes through the reagent channel 207. The matrix material may be a membrane including, for example, hydrophilic polyethersulfone, nitrocellulose, cellulose, or cellulose acetate membranes with a pore size of 0.1 to 10 microns. Reagents are preloaded or deposited into the matrix material 202. Reagent channel 207 opens into and is in fluid communication with a sample loading chamber 212. Sample is loaded into the sample loading chamber 212 through a sample inlet port 210 and a sample flow channel 211. The sample loading chamber is in fluid communication with an analysis or reaction chamber 214. Analysis chamber 214 is in fluid communication with a second vent channel 216 and a second vent port 218 which allows fluid movement into the analysis chamber 214 by preventing air blockages within the fluidic circuit by allowing air to vent out through the second vent port 218. A valve 208 may be placed between the solvent chamber 203 and the reagent channel 207 to prevent movement of solvent into the reagent channel when the disc is not in use. This also allows for the pre-loading of solvent or buffer into the solvent chamber for future use. The valve 208 may be, for example, a capillary valve. In use, a buffer such as PBS is loaded into the solvent chamber 203 while a sample such as whole blood is loaded into the sample loading chamber 212 through the sample inlet port 210 and the sample flow channel 211. The disc is then rotated using the optical disc drive 112 at a predetermined speed and time to allow the buffer to move into the reagent release channel 207 through the matrix material 202. Reagents deposited in the matrix material 202 are dissolved in the buffer when the buffer moves through the matrix material forming a reagent solution. The reagent solution then moves into the sample loading chamber and into the analysis chamber where it mixes with the blood sample. Since reagents are present in the reagent solution, analytes of interest that are present in the sample then react with the reagents to produce a detectable signal that is detected using the optical disc reader.

Referring next to FIG. 23A, there is illustrated an alternative embodiment of the fluidic circuit described in conjunction with FIG. 22. The fluidic circuit shown in FIG. 23A is herein referred to as a multi-solvent fluidic circuit wherein at least two different solvents may be pre-loaded in at least two solvent chambers. The solvents are mixed in a mixing channel 242 in the fluidic circuit. As would be apparent to one of skill in the art, the illustrated multi-solvent fluidic circuit may have more than two solvent chambers connected together by a solvent entry manifold. The multi-solvent fluidic circuit illustrated in FIG. 23A has two solvent chambers, a first solvent chamber 220 with a first solvent inlet port 224 and a second solvent chamber 222 with a second solvent inlet port 226. First solvent chamber 220 is in fluid communication with a first vent channel 228 which is in fluid communication with a first vent port 232 while the second solvent chamber 222 is in fluid communication with a second vent channel 230 which opens to a second vent port 234. The solvent chambers are connected to and in fluid communication with each other through a solvent entry manifold 240 which is in fluid communication with a first end of a mixing channel 242. Mixing channel 242 may be configured as a zigzag or sawtooth channel or stepwise channel with sharp angled edges, corners or turns as opposed to smooth non-angled channels wherein fluid flow is continuous with little or no turbulence. The mixing channels having angled edges mayenhances mixing of fluids in a fluidic circuit by creating turbulent flow. The path of mixing channel 242 is defined by a step function or a sawtooth function depending on the angle of the corners. The angle of the corners may be 5 to 160 degrees. As illustrated, fluid flow in the mixing channel is defined by a step function wherein the turns within the mixing channel are at 90 degree angles.

A second end of the mixing channel 242 is in fluid communication with one end of a reagent release chamber 207 having a reagent matrix material 202. The reagent release chamber 207 is connected to and is in fluid communication with a sample loading chamber 212. Sample is loaded into the sample loading chamber 212 through a sample inlet port 210; sample loaded though inlet port 210 flows into the sample loading chamber 212 through a sample flow channel 211. The sample loading chamber 212 is in fluid communication with an analysis chamber 214 which is fluidly connected to a third vent channel 244 with a third vent port 246. The vent channels 228, 230 and 244 along with the vent ports 232, 234 and 246 allow air to escape and prevent air blockages within the fluidic circuit. A first valve 236 may be optionally placed between the first solvent chamber 220 and the solvent entry manifold 240. A second valve 238 may also be placed between the second solvent chamber 222 and the solvent entry manifold 240. Valves 236 and 246 prevent entry of the solvents from the first and second solvent chambers into the rest of the multi-solvent fluidic circuit which allows pre-loading of solvents into the solvent chambers prior to use.

In one embodiment, solvents are pre-loaded into the solvent chambers and the disc is stored for future use. Depending on the type of solvent loaded, the disc may be stored at room temperature, or at 4° C., for example. For example, deionized water may be loaded into the first solvent chamber 220 while 2×PBS loaded into the second solvent chamber 222. The solvent inlet ports are sealed to prevent evaporation of the solvents and the disc is then stored at 4° C. The disc is taken out of storage and whole blood is loaded into the sample loading chamber 212 through the sample inlet port 210 and the sample flow channel 211. The disc is then rotated at a pre-determined speed and time to allow movement of the solvents (water and 2×PBS) into the entry manifold 240 and into the mixing chamber 242 where the solvents mix to produce an analysis buffer solution (e.g., 1×PBS from the dilution of 2×PBS). The analysis buffer then moves into the reagent channel 207 and through the reagent matrix material 202 where the reagents deposited within the matrix material 202 are released and dissolved into the analysis buffer as the buffer passes through the matrix material 202 producing a reagent buffer. The reagent buffer then mixes with the blood sample in the analysis chamber where the reagents react with analytes of interest that are present in the sample to produce a detectable signal or product.

Referring now to FIG. 23B, there is shown an alternate embodiment of the fluidic circuit described above in conjunction with FIG. 23A. The fluidic circuit illustrated in FIG. 23B is no longer a multi-solvent fluidic circuit since it only has a single solvent or buffer chamber 220 having a solvent or buffer inlet port 224. Solvent chamber 220 is in fluid communication with a reagent release channel or reagent source channel 207 having a reagent release area containing a reagent matrix material 202. Reagents for the detection of a pre-determined analyte may be deposited in the matrix material 202 as discussed above. The fluidic circuit shown in FIG. 23B also includes a sample loading chamber 212. Sample is loaded into the sample loading chamber 212 through a sample inlet port 210. Sample loading chamber 212 is in fluid communication with a sample flow channel 211. The reagent release channel 207 and the sample flow channel 211 are in fluid communication with each other and both connected to and in fluid communication with a sample-buffer mixing zone 209 as illustrated. The sample-buffer mixing zone 209 is in fluid communication with a first end 213 of a mixing channel 242 having a stepwise or switchback section 243 and a sawtooth section 245 with angled with angled corners to promote mixing of the sample and reagent buffer solution as they move through the mixing channel 242, in a radial direction, into an analysis chamber 214. Analysis chamber 214 is in fluid communication with a second end 215 of mixing chamber 242. Analysis chamber 214 is also in fluid communication with a vent channel 244 which opens to a vent port 246 to allow air to escape the fluidic circuit and prevent air blocks within the fluidic circuit. In use, for example, solvent or buffer such as PBS is loaded into the buffer chamber 220 through the buffer inlet port 224. A whole blood sample is also loaded into the sample chamber 212 through the sample inlet port 210. The disc is then rotated at a pre-determined speed and time to move the buffer into the reagent release channel 207 through the reagent matrix material 202 where the reagents deposited within the matrix material 202 are released and dissolved into the buffer as the buffer passes through the matrix material 202 producing a reagent buffer. While the buffer moves through the reagent release channel 207, the blood sample also moves through the sample flow channel 211. The blood sample then mixes with the reagent buffer at the sample-buffer mixing zone 209 to produce a reaction mixture. Mixing is further carried out while the reaction mixture (sample and buffer) moves through the mixing channel 242. While mixing in the mixing channel 242 the reagents in the buffer react with analytes of interest present in the sample to produce a detectable signal or product. The reaction mixture or suspension moves into the analysis chamber where further rotation of the disc causes the cells in the blood sample to pellet out of the suspension. The resulting detectable signal may then be investigated without interference from the cells using the optical disc drive 112.

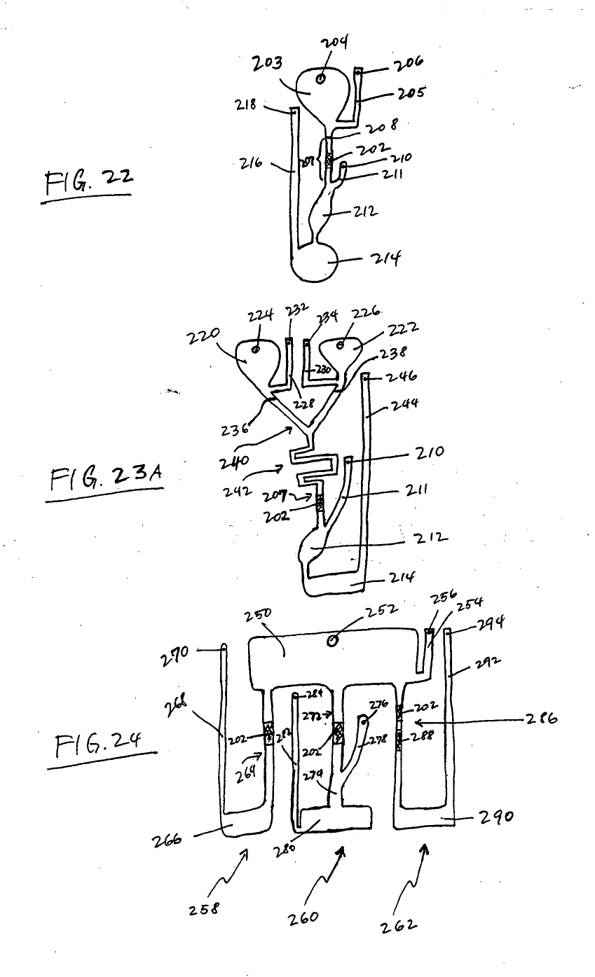

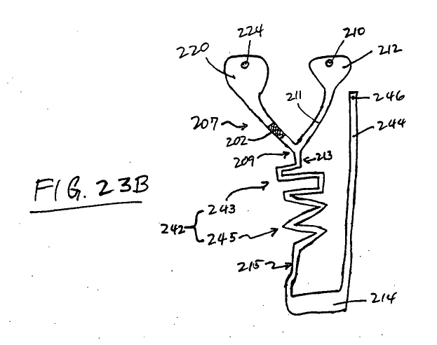

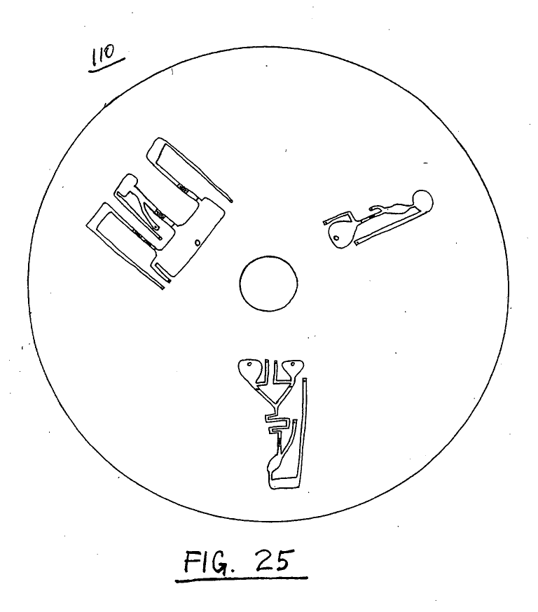

With reference now to FIG. 24, there is shown a fluidic circuit having multiple analysis fluidic circuits or analysis circuits including a negative control or reagent blank analysis circuit 258, an unknown or sample analysis circuit 260, and a positive control or maximum signal analysis circuit 262. The analysis circuits 258, 260, and 262 are in fluid communication with a solvent or buffer reservoir 250. Buffer is loaded into the reservoir 250 through a buffer inlet port 252. The buffer reservoir 250 is also in fluid communication with a first vent channel 254 having a vent port 256 that allows air to escape out of the reservoir 250 and prevent air blockages within reservoir 250. The reagent blank analysis circuit 258 is in fluid communication with the reservoir 250 through a first reagent release channel 264 which is in fluid communication with a first analysis chamber 266. The first analysis chamber 266 is in fluid communication with a first vent channel 268 having a first vent port 270 which allows entry of fluid from reservoir 250 into the first analysis chamber 266 and prevents air blocks within circuit 258. A reagent matrix material 202 is placed within the first reagent release channel 264. Similarly, the sample analysis circuit 260 is connected to and is in fluid communication with reservoir 250 through a second reagent release channel 272 also having reagent matrix material 202 placed therein. Channel 272 is connected to and in fluid communication with a sample loading chamber 274. Sample is loaded through a sample inlet port 276 into a sample flow channel 278 which is in fluid communication with the sample loading chamber 274. Loading chamber 274 is in fluid communication with and opens into a second analysis chamber 280 which is in fluid communication with a second vent channel 282 having a second vent port 284 which allows entry of fluid from reservoir 250 into the second analysis chamber 280 and prevents air blocks within sample analysis circuit 260. The maximum signal analysis circuit 262 is also in fluid communication with buffer reservoir 250 through a third reagent release channel 286. The reagent matrix material 202 is placed inside channel 286 and a substrate matrix material 288 is also placed within channel 286. As discussed above, one or more reagents used for analysis of one or more analytes is deposited in the reagent matrix material 202 prior to placing it into the reagent release channel; similarly, one or more substrates or analytes are loaded or deposited into the substrate matrix material 288 prior to placing it in the reagent release channel 286. Channel 288 opens into or is in fluid communication with a third analysis chamber 290 which is in fluid communication with a third vent channel 292 having a third vent port 294 which allows entry of fluid from reservoir 250 into the analysis chamber 290 and prevents air blocks within sample analysis circuit 262.

With continuing reference to FIG. 24, a buffer or solvent, such as PBS, is loaded into the buffer reservoir 250 through port 252. The disc may be stored prior to or after loading of buffer into reservoir 250. A sample, such as whole blood, is loaded into the sample loading chamber 274 through sample port 276 and sample flow channel 278. The disc is then rotated at a pre-determined speed and time to move the buffer in reservoir 250 into each of the reagent release channels 264, 272, and 286. Buffer then flows through each of the reagent matrix material 202 where the reagents deposited within the matrix material 202 are released and dissolved into the buffer as the buffer passes through the matrix material 202 producing a reagent buffer. The reagent buffer in channel 264 of the reagent blank analysis circuit 258 moves into the first analysis chamber 266. In the sample analysis circuit 260, while the buffer moves through the second reagent release channel 272, the blood sample also moves through the sample flow channel 278. The blood sample then mixes with the reagent buffer at the sample loading chamber 274. The reagents dissolved in the buffer react with analytes of interest present in the sample to produce a detectable signal or product. The final buffer and sample mixture or suspension moves into the second analysis chamber 280 where further rotation of the disc causes the cells in the blood sample to pellet out of the suspension. The resulting detectable signal may then be investigated without interference from the cells using the optical disc drive. Turning next to the maximum signal analysis circuit 262, buffer enters the third reagent release channel 286 and moves through the reagent matrix material 202 where the reagents deposited within the matrix material 202 are released and dissolved into the buffer as the buffer passes through material 202 producing a reagent buffer. The reagent buffer then moves through the substrate or analyte matrix material 288 dissolving the substrate or analyte deposited in material 288 producing a reaction solution. The reagents react with the substrate in the reaction solution producing a detectable signal. Further rotation of the disc causes the reagent solution to move into the third analysis chamber 290 for investigation using the optical bio-disc drive. The amount of substrate deposited in the substrate matrix material 288 is such that the reaction resulting from the mixture of the reagents and substrate produces the maximum level of signal for the reaction. The amount of analyte present in the sample is then calculated based on the signal collected from the respective analysis chambers of the reagent blank circuit 258 (no signal or 0% signal) and the maximum signal circuit 262 (100% signal). The fluidic circuits described above in conjunction with FIGS. 22-24 are shown in FIG. 25 as part of the bio-disc 110. The positions of the various components of the fluidic circuits, relative to the center of the disc, are also illustrated in FIG. 25.

Additional embodiments, aspects, details, and attributes of the invention are disclosed in Appendices A, B and C appended hereto. These appendices are therefore a part hereof wherein more specifically Appendix A includes pages A1-A30, Appendix B includes pages B1-B9, and Appendix C includes pages C1-C13

### CONCLUDING STATEMENTS

All patents, provisional applications, patent applications, and other publications mentioned in this specification are incorporated herein in their entireties by reference.

While this invention has been described in detail with reference to a certain preferred embodiments, it should be appreciated that the invention is not limited to those precise embodiments. Rather, in view of the present disclosure that describes the current best mode for practicing the invention, many modifications and variations would present themselves to those of skill in the art without departing from the scope and spirit of this invention. The scope of the invention is, therefore, indicated by the following claims rather than by the foregoing description. All changes, modifications, and variations coming within the meaning and range of equivalency of the claims are to be considered within their scope.

Furthermore, those skilled in the art will recognize, or be able to ascertain, using no more than routine experimentation, many equivalents to the specific embodiments of the invention described herein. Such equivalents are also intended to be encompassed by the following claims.

What is claimed is:

1. A fluidic circuit for analysis of a sample, comprising:
a reagent source channel having a first end and a second end;
a sample flow channel having a first end and a second end;
a buffer chamber having a buffer inlet port for receiving an amount of buffer, said buffer chamber in fluid communication with said first end of said reagent source channel;
a sample loading chamber having a sample inlet port for receiving samples, said sample loading chamber in fluid communication with said first end of said sample flow channel;
a mixing zone in fluid communication with said second end of said reagent source channel and said second end of said sample flow channel;
a mixing channel having a first and a second end, said first end of said mixing chamber in fluid communication with said mixing zone;
an analysis chamber in fluid communication with said second end of said mixing channel;
a vent channel in fluid communication with said analysis chamber; and
a vent port in fluid communication with said vent channel.

2. The fluidic circuit according to claim 1 wherein said mixing channel is configured as a switchback channel having corners that are at 90 degree angles to promote turbulent flow thereby enhancing mixing of fluids.

3. The fluidic circuit according to claim 1 wherein said mixing channel is in a sawtooth configuration having angled corners to promote turbulent flow thereby enhancing mixing of fluids.

4. The fluidic circuit according to claim 1 further comprising a reagent release area located within said reagent source channel.

5. The fluidic circuit according to claim 4 wherein reagents are deposited in said reagent release area.

6. The fluidic circuit according to claim 4 further comprising a reagent matrix material placed within said reagent release area.

7. The fluidic circuit according to claim 6 wherein reagents are deposited on said reagent matrix material.

8. An optical bio-disc for analysis of a sample comprising:
a substrate having encoded information associated therewith, said encoded information being readable by a disc drive assembly; and
a fluidic circuit associated with said substrate, said fluidic circuit comprising:
a reagent source channel having a first end and a second end;
a reagent matrix material formed within said reagent source channel;
a sample flow channel having a first and a second end;
a buffer chamber having a buffer inlet port for receiving an amount of buffer, said buffer chamber in fluid communication with said first end of said reagent source channel;
a sample loading chamber having a sample inlet port for receiving samples, said sample loading chamber in fluid communication with said first end of said sample flow channel;
a mixing zone in fluid communication with said second end of said reagent source channel and said second end of said sample flow channel;
a mixing channel having a first and a second end, said first end of said mixing chamber in fluid communication with said mixing zone;
an analysis chamber in fluid communication with said second end of said mixing channel;
a vent channel in fluid communication with said analysis chamber; and
a vent port in fluid communication with said vent channel.

9. A method for making an optical bio-disc for analysis of a sample, said method of making comprising the steps of:
providing a substantially circular substrate having encoded information associated therewith, said encoded information being readable by a disc drive assembly; and
providing a channel layer associated with said substrate;
providing a cap portion associated with said channel layer; and
forming a fluidic circuit within said channel layer, said fluidic circuit comprising:
a reagent source channel having a first end and a second end;
a reagent matrix material within said reagent source channel;
a sample flow channel having a first and a second end;
a buffer chamber for receiving an amount of buffer, said buffer chamber in fluid communication with said first end of said reagent source channel;
a buffer inlet port on said cap portion, said buffer inlet port in fluid communication with said buffer chamber;
a sample loading chamber for receiving samples, said sample loading chamber in fluid communication with said first end of said sample flow channel;
a sample inlet port on said cap portion, said inlet port in fluid communication with said sample loading chamber;
a mixing zone in fluid communication with said second end of said reagent source channel and said second end of said sample flow channel;
a mixing channel having a first and a second end, said first end of said mixing chamber in fluid communication with said mixing zone;
an analysis chamber in fluid communication with said second end of said mixing channel;
a vent channel in fluid communication with said analysis chamber; and
a vent port in said cap portion in fluid communication with said vent channel.

10. The method according to claim 9 further comprising the step of depositing reagents onto said reagent matrix material.

11. The method according to claim 10 further comprising the step of depositing a buffer into said buffer chamber.

12. A method of using an optical bio-disc comprising:
loading a sample into a sample loading chamber of a fluidic circuit;
placing said optical bio-disc into an optical disc drive;
reading an encoded information using said optical disc drive;
rotating said optical bio-disc to cause a buffer to move into a reagent release channel through a reagent matrix material thereby dissolving reagents deposited in said reagent matrix material producing a reagent buffer, said rotation also causes said sample to flow through a sample flow channel;
continuing said rotating step to further cause said reagent buffer and said sample to flow into said mixing zone and into said mixing chamber thereby mixing said sample and reagent buffer producing a reaction mixture;
continuing further said rotating step to cause the reaction mixture to move into said analysis chamber;
incubating said reaction mixture in said analysis chamber to allow said reagents to react with any analyte present in said sample to produce a detectable signal; and
scanning a beam of electromagnetic radiation through said analysis chamber using said optical disc drive to determine the presence and amount of said detectable signal.

13. The method according to claim 12 wherein said sample is a whole blood sample.

14. The method according to claim 13 wherein said whole blood sample is undiluted.

15. The method according to claim 12 wherein said buffer is selected from the group comprising sodium acetate, phosphate, Tris and PBS.

16. The method according to claim 12 wherein said reagent matrix material is selected from the group comprising hydrophilic polyethersulfone membrane, nitrocellulose, cellulose and cellulose acetate.

17. A fluidic circuit for analysis of a sample, comprising:
an analysis chamber;
a vent port in fluid communication with said vent channel.
a buffer chamber having a buffer inlet port for receiving an amount of buffer, said buffer chamber in fluid communication with said analysis chamber; and
a sample chamber having a sample inlet port for receiving samples, said sample loading chamber in fluid communication with said analysis chamber;

18. The fluidic circuit according to claim 17 further comprising a reagent release area located within said buffer chamber.

19. The fluidic circuit according to claim 18 wherein reagents are deposited in said reagent release area.

20. The fluidic circuit according to claim 18 further comprising a reagent matrix material placed within said reagent release area.

21. The fluidic circuit according to claim 20 wherein reagents are deposited on said reagent matrix material.

22. A method for performing an assay, comprising:
introducing a biological sample into a channel or reservoir in a bio-optical disk, wherein the bio-optical disk includes data or program information relevant to conducting or interpreting an assay for an analyte;
contacting the sample with one or more reagents that produce a first calorimetric signal in the presence of analyte in the sample;
contacting the said one or more reagents with a species that interacts with one or more of said reagents in competition with any analyte in the sample, wherein any colorimetric signal produced as a result of the presence of said species is spectrally distinguishable from the first colorimetric signal; and
measuring said first calorimetric signal to quantitate the amount of analyte, if any, in said sample.

23. The method of claim 22, wherein said species produces a second calorimetric signal in cooperation with said reagents, further comprising measuring said second colorimetric signal and comparing the magnitude thereof with said first calorimetric signal.

24. The method of claim 22, wherein said species produces a second calorimetric signal that is largely or wholly outside of a spectral range of sensitivity of a detector, such that said measuring step primarily or wholly involves measuring only said first calorimetric signal.

25. The method of claim 22, wherein said measuring step is performed in a disk drive and said species does not produce a signal that is substantially measured by said disk drive.

26. The method of claim 22, wherein one or more of said contacting steps is performed by moving fluid in said disk by spinning said disk at a predetermined speed.

27. The method of claim 22, wherein said disk includes computer-readable information relative to calibration.

28. The method of claim 22, wherein said disk includes computer-readable information that controls the performance of at least one aspect of the method, wherein the method is performed in a disk drive.
